(19) United States
(12) Patent Application Publication
(10) Pub. No.: US 2023/0165871 A1
(43) Pub. Date: Jun. 1, 2023

(54) COMBINATION THERAPIES FOR INHIBITION OF POLO-LIKE KINASE 4

(71) Applicant: UNIVERSITY HEALTH NETWORK, Toronto (CA)

(72) Inventors: Graham Fletcher, Toronto (CA); Jacqueline M. Mason, Toronto (CA); Mark R. Bray, Oakville (CA)

(21) Appl. No.: 17/917,027

(22) PCT Filed: Apr. 5, 2021
(86) PCT No.: PCT/CA2021/050452

§ 371 Date: Oct. 5, 2022

Related U.S. Application Data
(60) Provisional application No. 63/005,733, filed on Apr. 6, 2020.

(51) Int. Cl. A61K 31/5377; C07K 16/28; A61K 45/06

(52) U.S. Cl. CPC A61K 31/5377; A61K 45/06; C07K 16/2827

(57) ABSTRACT

Provided herein are methods of treating triple negative breast cancer using an effective amount of Compound (I) represented by the formula: or a pharmaceutically acceptable salt thereof and an effective amount of an immune checkpoint inhibitor, wherein the checkpoint inhibitor is a PD-1 inhibitor or a PD-L1 inhibitor. Uses of an effective amount of Compound [I] and an effective amount of an immune checkpoint inhibitor, wherein the checkpoint inhibitor is a PD-linhibitor or a PD-L1 inhibitor for treating triple negative breast cancer are also provided herein.

### CROSS-REFERENCE TO RELATED APPLICATIONS

This application claims priority to U.S. Provisional Application No. 63/005,733, filed Apr. 6, 2020. The entire contents of the aforementioned application are incorporated herein by reference.

### BACKGROUND

Recent advancements in the treatment of breast cancer have led to higher rates of cure for early stage disease, and longer survival for those living with metastatic disease. However, there remains a critical need for new and effective therapies for those whose disease is resistant or becomes resistant to currently available options. This need is especially urgent for triple negative breast cancer (TNBC), an aggressive subtype associated with early lethality, for which chemotherapy is the only approved treatment (apart from PARP inhibitors in patients with germline BRCA1/2 mutations).

PLK4 is an atypical member of the Polo-like serine/threonine kinases and differs from other PLK enzymes in structure and function. PLK4 controls centriole duplication and mitotic progression, and was identified as a drug target based on functional screening to identify vulnerabilities of genomically unstable breast cancers. Compound (I) is a potent, selective and orally bioavailable inhibitor of PLK4 (PLK4 Ki=0.26 nM), and was advanced into clinical development based on desirable pharmacologic properties and pre-clinical antitumour activity. Compound (I) is disclosed in Internation Patent Application Publication No. WO2010/115279, the entire contents of which are incorporated herein by reference.

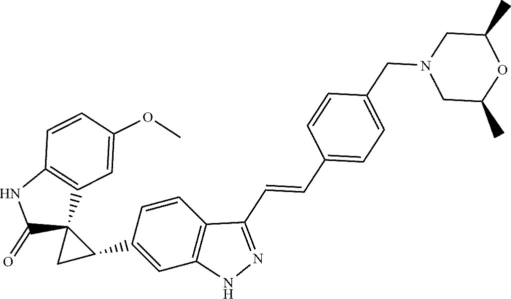 [SMILES: COc1ccc2c(c1)[C@]1(C[C@H]1c1ccc3c(/C=C/c4ccc(CN5C[C@@H](C)O[C@@H](C)C5)cc4)n[nH]c3c1)C(=O)N2]

In vitro, Compound (I) exhibits potent anti-proliferative effects in cancer cell lines via induction of apoptosis and cell cycle arrest with induction of aneuploidy. In vivo testing of diverse models, including cell line and patient-derived xenografts of breast and ovarian cancer confirmed potent anti-tumour effects at tolerable doses .

### SUMMARY

The present disclosure is directed towards treating triple negative breast cancer with Compound (I) or a pharmaceutically acceptable salt thereof and an immune checkpoint inhibitor , wherein the immune checkpoint inhibitor is a PD-1 inhibitor or a PD-L1 inhibitor.

Accordingly, provided herein are methods of treating triple negative breast cancer in a subject, by administering to the subject an effective amount of Compound (I) or a pharmaceutically acceptable salt thereof and an effective amount of immune checkpoint inhibitor as described herein.

### DETAILED DESCRIPTION

In one aspect, the present disclosure provides a method of treating triple negative breast cancer, comprising the step of administering to a subject in need thereof:
(i) an effective amount of Compound (I) represented by the formula:

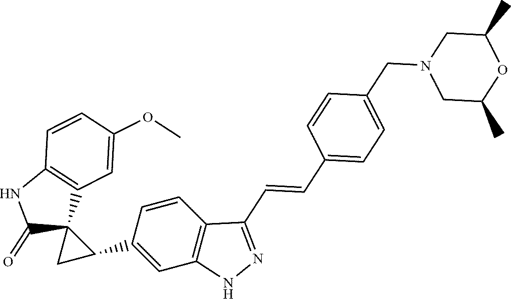 [SMILES: COc1ccc2c(c1)[C@]1(C[C@H]1c1ccc3c(/C=C/c4ccc(CN5C[C@@H](C)O[C@@H](C)C5)cc4)n[nH]c3c1)C(=O)N2]

or a pharmaceutically acceptable salt thereof; and
(ii) an effective amount of an immune checkpoint inhibitor wherein the immune checkpoint inhibitor is a PD-1 inhibitor or a PD-L1 inhibitor.

It will be understood that unless otherwise indicated, the administrations described herein include administering the described Compound (I) or a pharmaceutically acceptable salt thereof prior to, concurrently with, or after administration of the immune checkpoint inhibitor described herein. Thus, simultaneous administration is not necessary for therapeutic purposes. In one embodiment, however, the Compound (I) or a pharmaceutically acceptable salt thereof is administered concurrently with the immune checkpoint inhibitor.

The Compound (I) described herein has basic amine groups and therefore can form pharmaceutically acceptable salts with pharmaceutically acceptable acid(s). Accordingly, the term “pharmaceutically acceptable salt” as used herein refers to any suitable pharmaceutically acceptable acid addition salt of Compound (I) described herein, which includes but is not limited to salts of inorganic acids (e.g., hydrochloric acid, hydrobromic, phosphoric, nitric, and sulfuric acids) and of organic acids (such as, acetic acid, trifluoroacetic acid, fumaric, benzenesulfonic, benzoic, methanesulfonic, and p-toluenesulfonic acids). Other examples of such salts include hydrochlorides, hydrobromides, sulfates, methanesulfonates, nitrates, benzoates, trifluoroacetates, fumarates and salts with amino acids such as glutamic acid. In one embodiment, in the methods/pharmaceutical compositions disclosed therein, the subject in need thereof is administered fumarate salt of Compound (I). In a specific embodiment, in the methods/pharmaceutical compositions disclosed therein, the subject in need thereof is administered fumarate salt of Compound (I), wherein the molar ratio between compound (I) and fumaric acid is 1:1 (referred to herein as “1:1 fumarate salt of Compound (I)”).

As used herein, an “immune check point inhibitor” or simply a “checkpoint inhibitor” refers to any compound that, either directly or indirectly, decreases the level of or inhibits the function of an immune checkpoint receptor protein found on the surface of an immune cell (e.g., T-cells, B-cells, etc.). Alternatively, the immune checkpoint inhibitor is a compound that, either directly or indirectly, decreases the level of or inhibits the function of a ligand on the surface of an immune cell-inhibitory cell (e.g., regulatory T-cells, tolerogenic antigen presenting cells (APC), myeloid-derived suppressor cells (MDSC), tumor-associated macrophages (TAM), cancer-assicatied fibroblasts (CAF), other cancer cells and APCs, etc.), or secreted by an immune cell-inhibitory cell. This ligand is typically capable of binding the immune checkpoint receptor protein of the immune cell. A non-limiting example of an immune checkpoint receptor protein-ligand pair is PD-⅟PD-L1. PD-1 is an immune checkpoint receptor protein found on T-cells. PD-L1 that is often over-expressed by cancer cells binds to PD-1 and helps the cancer cells evade the host immune system attack. Accordingly, an immune checkpoint inhibitor prevents or reverses this PD-⅟PD-L1 binding, by either blocking the PD-1 on the T-cells (i.e., a PD-1 inhibitor) or the PD-L1 on the cancer cells (i.e., a PD-L1 inhibitor), thereby maintaining or restoring anti-tumor T-cell activity or blocking T-cell-inhibitory cell activity. Additionally, an immune checkpoint inhibitor refers to a compound as described in U.S. Pat. Application Publication Nos. US 2017/0190675 and US 2016/0185870, and International Patent Application Publication Nos. WO 2015/112900, WO 2010/027828 and WO 2010/036959, the entire teachings of which are incorporated herein by reference.

An immune checkpoint inhibitor in accordance with the present disclosure may be a PD-1 inhibitor or a PD-L1 inhibitor. In one embodiment, the immune checkpoint inhibitor is a PD-1 inhibitor. In another embodiment, the immune checkpoint inhibitor is a PD-L1 inhibitor.

In some embodiments, the immune checkpoint inhibitor is a PD-1 inhibitor selected from pembrolizumab, nivolumab, cemiplimab (REGN2810), spartalizumab (PDR001), camrelizumab (SHR1210), Sintilimab (IBI308), Tislelizumab (BGB-A317), Toripalimab (JS001), Nivolumab (BMS-936558), AMP-224, and AMP-514. In a specific embodiment, the PD-1 inhibitor is pembrolizumab, nivolumab, or cemiplimab (REGN2810).

In some embodiments, the immune checkpoint inhibitor is a PD-L1 inhibitor selected from atezolizumab, avelumab, durvalumab, JS003, KN035, CK-301, AUNP12, CA-170, BMS-936559, BMS-986189, and SHR-1316. In a specific embodiment, the PD-L1 inhibitor is atezolizumab, avelumab, or durvalumab (e.g., durvalumab).

As used herein, the terms “treatment,” “treat,” and “treating” refer to reversing, alleviating, ameliorating, inhibiting or slowing the progression of a cancer, reducing the likelihood of recurrence of a cancer, or one or more symptoms thereof, as described herein.

The term “triple negative breast cancer” refers to a breast cancer that tests negative for hormones estrogen, progesterone, and HER2 under the ASCO/CAP Clinical Practice Guideline. In some embodiments, the triple negative breast cancer is unresectable or metastatic.

The term “an effective amount” means an amount when administered to a subject which results in beneficial or desired results, including clinical results, i.e., reversing, alleviating, inhibiting or slowing the progression of a cancer (i.e., triple negative breast cancer), reducing the likelihood of recurrence of a cancer, or one or more symptoms thereof, e.g., as determined by clinical symptoms, the amount or volume or cancer cells or tumors in a subject compared to a control.

In an aspect, an effective amount of Compound (I) or a pharmaceutically acceptable salt thereof disclosed herein ranges from about 0.1 to about 1000 mg/kg body weight, alternatively about 1 to about 500 mg/kg body weight, and in another alternative, from about 1 to about 100 mg/kg body weight, and in yet another alternative, from about 1 to about 50 mg/kg, and in yet another alternative, from about 0.1 to about 10 mg/kg body weight, and in yet another alternative from about 1 to about 7 mg/kg body weight or about 1 to about 6.5 mg/kg body weight if administered daily. In an embodiment, an effective amount of an immune checkpoint inhibitor taught herein ranges from about 0.01 to about 1000 µg/kg body weight, alternatively from about 0.05 to about 500 µg/kg body weight. The skilled artisan will appreciate that certain factors may influence the dosage required to effectively treat a subject suffering from cancer or reduce the likelihood of recurrence of a cancer. These factors include, but are not limited to, the classification and/or severity of the disease or disorder, previous treatments, the general health and/or age of the subject and other diseases present.

In some embodiments, the subject in need thereof is administered Compound (I) in an amount of 3 mg to 96 mg (e.g., once a day), or a pharmaceutically acceptable salt thereof in an amount equivalent to 3 mg to 96 mg of Compound (I) (e.g., once a day).

In some embodiments, the subject in need thereof is administered Compound (I) in an amount of 32 mg to 64 mg (e.g., once a day), or a pharmaceutically acceptable salt thereof in an amount equivalent to 32 mg to 64 mg of Compound (I) (e.g., once a day).

In a specific embodiment, the subject in need thereof is administered Compound (I) in an amount of 10 mg (e.g., once a day), or a pharmaceutically acceptable salt thereof in an amount equivalent to 10 mg of Compound (I) (e.g., once a day).

In a specific embodiment, the subject in need thereof is administered Compound (I) in an amount of 16 mg (e.g., once a day), or a pharmaceutically acceptable salt thereof in an amount equivalent to 16 mg of Compound (I) (e.g., once a day).

In a specific embodiment, the subject in need thereof is administered Compound (I) in an amount of 20 mg (e.g., once a day), or a pharmaceutically acceptable salt thereof in an amount equivalent to 20 mg of Compound (I) (e.g., once a day).

In a specific embodiment, the subject in need thereof is administered Compound (I) in an amount of 24 mg (e.g., once a day), or a pharmaceutically acceptable salt thereof in an amount equivalent to 24 mg of Compound (I) (e.g., once a day).

In a specific embodiment, the subject in need thereof is administered Compound (I) in an amount of 30 mg (e.g., once a day), or a pharmaceutically acceptable salt thereof in an amount equivalent to 30 mg of Compound (I) (e.g., once a day).

In a specific embodiment, the subject in need thereof is administered Compound (I) in an amount of 32 mg (e.g., once a day), or a pharmaceutically acceptable salt thereof in an amount equivalent to 32 mg of Compound (I) (e.g., once a day).

In a specific embodiment, the subject in need thereof is administered Compound (I) in an amount of 40 mg (e.g., once a day), or a pharmaceutically acceptable salt thereof in an amount equivalent to 40 mg of Compound (I) (e.g., once a day).

In a specific embodiment, the subject in need thereof is administered Compound (I) in an amount of 48 mg (e.g., once a day), or a pharmaceutically acceptable salt thereof in an amount equivalent to 48 mg of Compound (I) (e.g., once a day).

In a specific embodiment, the subject in need thereof is administered Compound (I) in an amount of 50 mg (e.g., once a day), or a pharmaceutically acceptable salt thereof in an amount equivalent to 50 mg of Compound (I) (e.g., once a day).

In a specific embodiment, the subject in need thereof is administered Compound (I) in an amount of 56 mg (e.g., once a day), or a pharmaceutically acceptable salt thereof in an amount equivalent to 56 mg of Compound (I) (e.g., once a day).

In a specific embodiment, the subject in need thereof is administered Compound (I) in an amount of 60 mg (e.g., once a day), or a pharmaceutically acceptable salt thereof in an amount equivalent to 60 mg of Compound (I) (e.g., once a day).

In a specific embodiment, the subject in need thereof is administered Compound (I) in an amount of 64 mg (e.g., once a day), or a pharmaceutically acceptable salt thereof in an amount equivalent to 64 mg of Compound (I) (e.g., once a day).

In a specific embodiment, the subject in need thereof is administered Compound (I) in an amount of 70 mg (e.g., once a day), or a pharmaceutically acceptable salt thereof in an amount equivalent to 70 mg of Compound (I) (e.g., once a day).

In a specific embodiment, the subject in need thereof is administered Compound (I) in an amount of 72 mg (e.g., once a day), or a pharmaceutically acceptable salt thereof in an amount equivalent to 72 mg of Compound (I) (e.g., once a day).

In a specific embodiment, the subject in need thereof is administered Compound (I) in an amount of 80 mg (e.g., once a day), or a pharmaceutically acceptable salt thereof in an amount equivalent to 80 mg of Compound (I) (e.g., once a day).

In a specific embodiment, the subject in need thereof is administered Compound (I) in an amount of 96 mg (e.g., once a day), or a pharmaceutically acceptable salt thereof in an amount equivalent to 96 mg of Compound (I) (e.g., once a day).

In some embodiments, the subject in need thereof is administered an effective amount of Compound (I) or a pharmaceutically acceptable salt thereof 1, 2, 3, 4, 5, 6, or 7 times every week. In some embodiments, Compound (I) or a pharmaceutically acceptable salt thereof is administered continuously for at least 1 week, at least 2 weeks, at least 3 weeks, at least 4 weeks, at least 5 weeks, at least 6 weeks, at least 7 weeks, or at least 8 weeks.

In some embodiments, Compound (I) or a pharmaceutically acceptable salt thereof is administered in a cycle of once daily for a week and then a week off, wherein the cycle is repeated at least once. In some embodiments, the cycle is repeated at least twice, at least three times, at least four times, at least five times, at least six times, at least sevent times, at least eight times, at least nine times, at least ten times.

In some embodiments, the immune checkpoint inhibitor (a PD-1 inhibitor or a PD-L1 inhibitor) is administered in an amount of about 0.005 mg to about 5000 mg every week, every 2 weeks, every 3 weeks, or every 4 weeks, such as about 0.005, 0.05, 0.5, 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg every week, every 2 weeks, every 3 weeks, or every 4 weeks.

In some embodiments, the immune checkpoint inhibitor (a PD-1 inhibitor or a PD-L1 inhibitor) is administered in an amount of about 1 ng/kg to about 200 mg/kg, about 1 µg/kg to about 100 mg/kg, or about 1 mg/kg to about 50 mg/kg per unit dose, such as administered in an amount of about 1 µg/kg, about 10 µg/kg, about 25 µg/kg, about 50 µg/kg, about 75 µg/kg, about 100 µg/kg, about 125 µg/kg, about 150 µg/kg, about 175 µg/kg, about 200 µg/kg, about 225 µg/kg, about 250 µg/kg, about 275 µg/kg, about 300 µg/kg, about 325 µg/kg, about 350 µg/kg, about 375 µg/kg, about 400 µg/kg, about 425 µg/kg, about 450 µg/kg, about 475 µg/kg, about 500 µg/kg, about 525 µg/kg, about 550 µg/kg, about 575 µg/kg, about 600 µg/kg, about 625 µg/kg, about 650 µg/kg, about 675 µg/kg, about 700 µg/kg, about 725 µg/kg, about 750 µg/kg, about 775 µg/kg, about 800 µg/kg, about 825 µg/kg, about 850 µg/kg, about 875 µg/kg, about 900 µg/kg, about 925 µg/kg, about 950 µg/kg, about 975 µg/kg, about 1 mg/kg, about 1.5 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, and about 200 mg/kg per unit dose, and one or more (e.g., 1, 2, 3, 4, 5, 6, 7, 8, 9, 10, 15 or 20) unit doses are administered every week, every 2 weeks, every 3 weeks, or every 4 weeks.

In some embodiments, the subject in need thereof is administered the checkpoint inhibitor once every week, every 2 weeks, every 3 weeks, or every 4 weeks. In a specific embodiment, the immune checkpoint inhibitor is a PD-L1 inhibitor (e.g., durvalumab). In one specific embodiment, the subject in need thereof is administered PD-L1 inhibitor (e.g., durvalumab) in an amount of 1500 mg when the subject is > 30 kg in weight or in an amount of 20 mg/kg when the subject is ≤ 30 kg. In another specific embodiment, the subject in need thereof is administered PD-L1 inhibitor (e.g., durvalumab) in an amount of 5 mg/kg every 4 weeks. In another specific embodiment, the subject in need thereof is administered PD-L1 inhibitor (e.g., durvalumab) in an amount of 10 mg/kg every 4 weeks. In another specific embodiment, the subject in need thereof is administered PD-L1 inhibitor (e.g., durvalumab) in an amount of 15 mg/kg every 4 weeks. In another specific embodiment, the subject in need thereof is administered PD-L1 inhibitor (e.g., durvalumab) in an amount of 20 mg/kg every 4 weeks. In another specific embodiment, the subject in need thereof is administered PD-L1 inhibitor (e.g., durvalumab) in an amount of 25 mg/kg every 4 weeks. In another specific embodiment, the subject in need thereof is administered PD-L1 inhibitor (e.g., durvalumab) in an amount of 30 mg/kg every 4 weeks. In another specific embodiment, the subject in need thereof is administered PD-L1 inhibitor (e.g., durvalumab) in an amount of 40 mg/kg every 4 weeks. In another specific embodiment, the subject in need thereof is administered PD-L1 inhibitor (e.g., durvalumab) in an amount of 45 mg/kg every 4 weeks. In another specific embodiment, the subject in need thereof is administered PD-L1 inhibitor (e.g., durvalumab) in an amount of 50 mg/kg every 4 weeks. In another specific embodiment, the subject in need thereof is administered PD-L1 inhibitor (e.g., durvalumab) in an amount of 55 mg/kg every 4 weeks. In another specific embodiment, the subject in need thereof is administered PD-L1 inhibitor (e.g., durvalumab) in an amount of 60 mg/kg every 4 weeks.

In certain embodiments, Compound (I) or a pharmaceutically acceptable salt thereof, and/or the immune checkpoint inhibitor (a PD-1 inhibitor or a PD-L1 inhibitor) are administered for one or more (e.g., 1, 2, 3, 4, 5, 6, 7, 8, 9 or 10) courses of treatment, wherein each course of treatment lasts for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days or at least 50 days, at least 2 weeks, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks; wherein for each course of treatment, administration is performed 1, 2, 3, 4, 5, 6, 7, 8, 9 or 10 times; and the interval between every two courses of treatment is 0, 1, 2, 3, 4, 5, 6, 7, 8, 9, 10 days, 2 weeks, 3 weeks, 4 weeks, 1 month or 2 months.

Also included are the use of Compound (I) or a pharmaceutically acceptable salt thereof in the manufacture of a medicament to be used in combination with an immune checkpoint inhibitor as described herein for the treatment of triple negative breast cancer described herein. Also included herein are pharmaceutical compositions comprising Compound (I) or a pharmaceutically acceptable salt thereof and an immune checkpoint inhibitor as described herein optionally together with a pharmaceutically acceptable carrier, in the manufacture of a medicament for the treatment of triple negative breast cancer described herein. Also included is Compound (I) for use in combination with an immune checkpoint inhibitor as described herein for the treatment of a subject with triple negative breast cancer. Further included are pharmaceutical compositions comprising Compound (I) or a pharmaceutically acceptable salt thereof and an immune checkpoint inhibitor as described herein, optionally together with a pharmaceutically acceptable carrier, for use in the treatment of triple negative breast cancer described herein. Further included are pharmaceutical compositions comprising Compound (I) or a pharmaceutically acceptable salt thereof and an immune checkpoint inhibitor as described herein optionally together with a pharmaceutically acceptable carrier for use in the treatment of triple negative breast cancer.

The term “pharmaceutically acceptable carrier” refers to a non-toxic carrier, diluent, adjuvant, vehicle or excipient that does not adversely affect the pharmacological activity of the compound with which it is formulated, and which is also safe for human use. Pharmaceutically acceptable carriers that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat.

Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5th Ed., a Pharmaceutical Press (2005)). A person skilled in the art would know how to prepare formulations suitable for various types of administration routes. Conventional procedures and ingredients for the selection and preparation of suitable formulations are described, for example, in Remington’s Pharmaceutical Sciences (2003, 20th edition) and in The United States Pharmacopeia: The National Formulary (USP 24 NF19) published in 1999.

Compound (I) or a pharmaceutically acceptable salt thereof and the immune checkpoint inhibitor, or the compositions of the present teachings may be administered, for example, by oral, parenteral, sublingual, topical, rectal, nasal, buccal, vaginal, transdermal, patch, pump administration or via an implanted reservoir, and the pharmaceutical compositions would be formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

Other forms of administration included in this disclosure are as described in WO 2013/075083, WO 2013/075084, WO 2013/078320, WO 2013/120104, WO 2014/124418, WO 2014/151142, WO 2015/023915, and WO 2019/046949, the contents of which are incorporated herein by reference.

### EXEMPLIFICATION

While we have described a number of embodiments of this invention, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

### Example 1

This is a multicentre, open-label phase II study of Compound (I) and durvalumab in patients with advanced or metastatic triple negative breast cancer. Up to 28 patients will be enrolled.

| | | | | |
|---|---|---|---|---|
| Patient with unresectable locally recurrent or metastatic triple negative breast cancer | ➔ | Cycle 1 Compound (I) orally on Days 1 to 7 (then 7 days off) and on Days 15 to 21 (then 7 days off) | ➔ | Cycle 2 on: Compound (I) orally once daily x 28 days and Durvalumab 1500 mg IV on Day 1 q28d |

The study will (i) evaluate the objective response rate (Response Evaluation Criteria in Solid Tumours , RECIST 1.1) of Compound (I) given with durvalumab, (ii) evaluate Disease Control Rate (DCR, defined as complete response, i.e., CR or partial response, i.e., PR or stable disease (SD) > 16 weeks in duration) of Compound (I) given with durvalumab; (iii) evaluate the immune-related response rate (iRECIST) of Compound (I) given with durvalumab; (iv) establish the safety and tolerability of Compound (I) given orally in combination with durvalumab in a q4w schedule and to confirm the recommended phase II dose (RP2D) in patients with metastatic triple negative breast cancer (TNBC); and (v) assess the pharmacodynamic and immune effects of Compound (I)+durvalumab.

Patients must have histologically and/or cytologically confirmed diagnosis of breast cancer, that is advanced/metastatic or unresectable, for which no curative therapy exists, and be negative for ER, PR and HER2 by ASCO/CAP criteria on the most recent sample. Patients with tumour with either low (< 10%) ER expression who are PR and HER2 negative, or ER and HER2 negative but with low PR (< 10%) may be enrolled after discussion and confirmation with regulatory sponsor.

Patients must have had at least 1 prior line of cytotoxic chemotherapy for breast cancer, in any setting, which must have included an anthracycline and a taxane (unless contraindicated). There is no limit to the number of prior chemotherapy regimens.

### Treatment Plan

No pharmacokinetic or pharmacodynamic interaction is expected to occur and given the non- overlapping toxicity profiles, no formal dose escalation phase will occur. A safety review after the enrollment of 6 patients, who have been followed for cycles 1-3, will be conducted prior to reopening to further accrual. If needed, the starting dose of Compound (I) will be reduced by one dose level, or if other trials suggest a different RP2D, changed to the new RP2D.

Drug Administration

| Cycle | Agents | Dose | Route | Duration | Schedule | Cycle Duration (days) |
|---|---|---|---|---|---|---|
| 1 | Compound (I) | Assigned at enrollment | Oral | - | Days 1-7, 15-21 | 28 |
| 2 onwards | | | | - | Daily^{∗} | 28 |
| 2 onwards | Durvalumab | 1500 mg | IV | 60 min | Day 1 | 28 |

^{∗}An intermittent schedule may be used, see section dose levels below

Cycle 1 will consist of Compound (I) administered at the starting dose assigned at enrollment on a one week on and one week off schedule. Depending on the toxicity observed in Cycle 1, Cycle 2 Day 1 will begin with durvalumab administration and Compound (I) at either full or reduced dose (Section 7.1.4 Table 1) continuously in subsequent 28-day repeating treatment cycles. Patients who experience no myelosuppression in 2 consecutive cycles may have Compound (I) dose escalated to 64 mg (see 7.1.3). Patients may have the Compound (I) dose reduced at any time due to toxicity. If either agent is held for toxicity, the other agent should also be held unless the toxicity is neutropenia alone.

Patients will take Compound (I) by mouth at least 1 hour before, and 2 hours after, any food and should be encouraged to take the Compound (I) at the same time each day at the time they find most convenient and are least likely to forget. On days when both Compound (I) and durvalumab are administered, the timing of durvalumab administration in relation to Compound (I) is not important, as long as they are started on the same day.

### Dose Levels

The following are dose levels for Compound (I), which will be titrated according to the degree of toxicity:

| | | | | |
|---|---|---|---|---|
| DL1 | DL-1 | DL-2 | DL-3 | DL2-escalation |
| 48 mg daily | 40 mg daily | 32 mg daily | 32 mg / 5 days per week (5 days on/2 days off) | 64 mg daily |

Durvalumab will be administered as a flat dose of 1500 mg for patients > 30 kg in weight. If patient weight falls to ≤ 30 kg, weight-based dosing at 20 mg/kg will be administered. No dose reductions are planned.

### Dose Modifications - Compound (I)

Doses will be reduced for hematologic and other adverse events. Dose adjustments are to be made according to the system showing the greatest degree of toxicity. Adverse events will be graded using the NCI Common Terminology Criteria for Adverse Events (CTCAE).

The major toxic effects of Compound (I) which limit dose are neutropenia, thrombocytopenia, fever, diarrhea and increased liver function tests. The guidelines which follow outline dose adjustments for several of these toxic effects. If a patient experiences several adverse events and there are conflicting recommendations, please use the recommended dose adjustment that reduces the dose to the lowest level. Doses should only be modified for toxicity related to Compound (I).

Diarrhea > Grade 1 is not a common toxicity of Compound (I). Patients who experience diarrhea should be carefully evaluated for possible immune mediated colitis and managed accordingly.

Following any interruption of dosing for toxicity as described below, Compound (I) should not be re- started until ANC ≥ 1.5 x 109/L and platelets ≥ 100 x 109/L and resolution of all drug related toxicity to ≤ grade 2.

### Dose Adjustments - Durvalumab

The major toxic effects of durvalumab which are anticipated to limit dosing are hypersensitivity/ infusion related reactions and possible class related immune related AEs, based on the mechanism of action of durvalumab leading to T-cell activation and proliferation. Potential immune related AEs across the class include pneumonitis, hepatitis, diarrhea/colitis and intestinal perforation, endocrinopathies (hypo and hyperthyroidism, adrenal insufficiency, hypophysitis/hypopituitarism diabetes insipidus and Type 1 diabetes mellitus), nephritis, rash/dermatitis, pancreatitis, myocarditis, myositis/polymyositis and rare/less frequent irAEs including neuromuscular toxicities such as myasthenia gravis and Guillain-Barre syndrome. Other inflammatory responses that are rare with a potential immune-mediated etiology include, but are not limited to, pericarditis, sarcoidosis, uveitis and other events involving the eye (e.g. keratitis and optic neuritis), skin (e.g. scleroderma, vitiligo and pemphigoid), hematological (e.g. hemolytic anemia and immune thrombocytopenic purpura) and rheumatological (e.g. polymyalgia rheumatic and autoimmune arthritis) events, vasculitis, non infectious encephalitis or non infectious meningitis. It is possible that events with an inflammatory or immune mediated mechanism could occur in nearly all organs.

If a patient experiences several adverse events and there are conflicting recommendations, please use the recommended dose adjustment that requires the greatest dose hold or discontinuation. Adverse events will be graded using the NCI Common Terminology Criteria for Adverse Events (CTCAE).

Dose adjustments (slowing/interruption of infusion rate, omission of a dose, or permanent discontinuation) will be made for durvalumab- related adverse events.

If the infusion cannot be administered, it should be omitted until the next planned infusion.

The next cycle should not be given until the laboratory criteria are met and resolution of all drug related toxicity to ≤ grade 2. For patients who have discontinued Compound (I) and are continuing on durvalumab alone, criteria for creatinine clearance may be lower.

### Criteria for Measurement of Study Endpoints

### Evaluable for Adverse Events

All patients will be evaluable for adverse event evaluation from the time of their first treatment.

### Evaluable for Response

All patients who have received at least one cycle 1 of therapy and have their disease re-evaluated will be considered evaluable for response (exceptions will be those who exhibit objective disease progression prior to the end of cycle 1 who will also be considered evaluable). Patients on therapy for at least this period and who meet the other listed criteria will have their response classified according to the definitions set out below (Eisenhauer E, et al. New response evaluation criteria in solid tumors: Revised RECIST guideline version 1.1. Eur J Can 45: 228-47, 2009).

Response and progression will be evaluated in this study using the revised international criteria (1.1) proposed by the RECIST (Response Evaluation Criteria in Solid Tumours) committee as well as the modified iRECIST guidelines (Seymour et al. Guidelines for response criteria for use in trials testing immunotherapeutics. Lancet Oncol. 2017;18:143-52).

### Response and Evaluation Endpoints

Response and progression will be evaluated in this study using the revised international criteria (1.1) proposed by the RECIST (Response Evaluation Criteria in Solid Tumours) committee as well as the Immune-Related modified RECIST. Investigators should continue treatment, as appropriate, in the absence of unacceptable toxicity, until unequivocal disease progression. This is particularly important for patients in whom pseudoprogression may have occurred. Follow up response assessments must be continued until unequivocal disease progression has occurred.

### Measurable Disease

Measurable tumour lesions are defined as those that can be accurately measured in at least one dimension (longest diameter to be recorded) as ≥ 20 mm with chest x-ray and as ≥ 10 mm with CT scan or clinical examination. Bone lesions are considered measurable only if assessed by CT scan and have an identifiable soft tissue component that meets these requirements (soft tissue component ≥ 10 mm by CT scan). Malignant lymph nodes must be ≥ 15 mm in the short axis to be considered measurable; only the short axis will be measured and followed. All tumour measurements must be recorded in millimetres (or decimal fractions of centimetres). Previously irradiated lesions are not considered measurable unless progression has been documented in the lesion.

### Non-Measurable Disease

All other lesions (or sites of disease), including small lesions are considered non-measurable disease. Bone lesions without a measurable soft tissue component, leptomeningeal disease, ascites, pleural/pericardial effusions, lymphangitis cutis/pulmonis, inflammatory breast disease, lymphangitic involvement of lung or skin and abdominal masses followed by clinical examination are all non-measurable. Lesions in previously irradiated areas are non-measurable, unless progression has been demonstrated.

### Target Lesions

When more than one measurable tumour lesion is present at baseline all lesions up to a maximum of 5 lesions total (and a maximum of 2 lesions per organ) representative of all involved organs should be identified as target lesions and will be recorded and measured at baseline. Target lesions should be selected on the basis of their size (lesions with the longest diameter), be representative of all involved organs, but in addition should be those that lend themselves to reproducible repeated measurements. Note that pathological nodes must meet the criterion of a short axis of ≥ 15 mm by CT scan and only the short axis of these nodes will contribute to the baseline sum. All other pathological nodes (those with short axis ≥ 10 mm but < 15 mm) should be considered non-target lesions. Nodes that have a short axis < 10 mm are considered non- pathological and should not be recorded or followed (see 8.2.4). At baseline, the sum of the target lesions (longest diameter of tumour lesions plus short axis of lymph nodes: overall maximumof 5) is to be recorded.

After baseline, a value should be provided on the CRF for all identified target lesions for each assessment, even if very small. If extremely small and faint lesions cannot be accuratelymeasured but are deemed to be present, a default value of 5 mm may be used. If lesions are too small to measure and indeed are believed to be absent, a default value of 0 mm may be used.

### Non-Target Lesions

All non-measurable lesions (or sites of disease) plus any measurable lesions over and above those listed as target lesions are considered non-target lesions. Measurements are not required but these lesions should be noted at baseline and should be followed as “present” or “absent”.

### Response

All patients will have their BEST RESPONSE from the start of study treatment until the end of treatment classified as outlined below:

Complete Response (CR): disappearance of target and non-target lesions and normalization of tumour markers. Pathological lymph nodes must have short axis measures < 10 mm (Note: continue to record the measurement even if < 10 mm and considered CR). Residual lesions (other than nodes < 10 mm) thought to be non-malignant should be further investigated (by cytology specialized imaging or other techniques as appropriate for individual cases [Eisenhauer 2009]) before CR can be accepted. Confirmation of response is required.

Partial Response (PR): at least a 30% decrease in the sum of measures (longest diameter for tumour lesions and short axis measure for nodes) of target lesions, taking as reference the baseline sum of diameters. Non target lesions must be non-PD. Confirmation of response is required.

Stable Disease (SD): Neither sufficient shrinkage to qualify for PR nor sufficient increase to qualify for PD taking as reference the smallest sum of diameters on study.

Progressive Disease (PD): at least a 20% increase in the sum of diameters of measured lesions taking as references the smallest sum of diameters recorded on study (including baseline) ANDan absolute increase of ≥ 5 mm. Appearance of new lesions will also constitute progressive disease (including lesions in previously unassessed areas). In exceptional circumstances, unequivocal progression of non-target disease may be accepted as evidence of disease progression, where the overall tumour burden has increased sufficiently to merit discontinuation of treatment or where the tumour burden appears to have increased by at least 73% in volume. Modest increases in the size of one or more non-target lesions are NOT considered unequivocal progression. If the evidence of PD is equivocal (target or non-target), treatment may continue until the next assessment, but if confirmed, the earlier date must be used.

### Immune-Related Response Assessment

Overall response will also be assessed using iRECIST [Seymour 2017]. Immunotherapeutics may result in infiltration of immune cells leading to transient increase in the size in malignant lesions, or undetectable lesions becoming detectable. The criteria are identical to those of RECIST 1.1 in many respects but have been adapted to account for instances where an increase in tumour burden, or the appearance of new lesions, does not reflect true tumour progression.

Key differences are described below. All responses defined using iRECIST criteria are designated with a prefix. iRECIST time-point and best overall responses will be recorded separately.

### Confirming Progression

Unlike RECIST 1.1, iRECIST requires the confirmation of progression and uses the terms iUPD (unconfirmed progression) and iCPD (confirmed progression). Confirmatory scans should be performed at least 4 weeks, but no longer than 8 weeks after iUPD.

iCPD is confirmed if further increase in tumour burden, compared to the last assessment, is seen as evidenced by one or more of the following:
Continued increase in tumour burden (from iUPD) where RECIST 1.1 definitions of progression had been met (fromnadir) in target, non-target disease or new lesions - Progression in target disease worsens with an increase of at least 5 mm in the absolute value of thesum:
   Continued unequivocal progression in non-target diseasewith an increase in tumour burden;
   Increase in size of previously identified new lesion (s) (an increase of at least 5 mm in the absolute value of the sum of those considered to be target new lesions) or additional new lesions.
RECIST 1.1 criteria are met in lesions types (target or non-target or new lesions) where progression was not previously identified, including the appearance of additional newlesions.

If iUPD is not confirmed at the next assessment, then the appropriate response will be assigned (iUPD if the criteria are still met, but no worsening, or iSD, iPR or iCR if those criteria are met compared to baseline). As can be seen in table 2, the prior documentation of iUPD does not preclude assigning iCR, iPR, or iSD in subsequent time-point assessments or as best overall response (BOR) providing that iCPD is not documented at the next assessment after iUPD.

### New Lesions

New lesions should be assessed and measured as they appear using RECIST 1.1 criteria (maximum of 5 lesions, no more than 2 per site, at least 10 mm in long axis (or 15 mm in short axis for nodal lesions), and recorded as New Lesions-Target (NLT) and New Lesion-Non-Target (NLNT) to allow clear differentiation from baseline target and non-target lesions.

New lesions may either meet the criteria of NLT or NLNT to drive iUPD (or iCPD). However, the measurements of NLT should NOT be included in the sum of measures of original target lesions identified at baseline. Rather, these measurements will be collected on a separate table in the case record form.

PD is confirmed in the New Lesion category if the next imaging assessment, conducted at least 4 weeks (but not more than 8 weeks) after iUPD confirms further progression fromiUPD with either an increase of at least 5 mm in the absolute value of the sum of NLT OR an increase (but not necessarily unequivocal increase) in the size of NLNT lesions OR the appearance of additional new lesions.

1. A method for treating triple negative breast cancer, comprising administering to a subject in need thereof:
(i) an effective amount of Compound (I) represented by the formula: 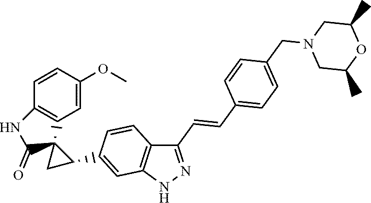 [SMILES: COc1ccc2c(c1)[C@]1(C[C@H]1c1ccc3c(/C=C/c4ccc(CN5C[C@@H](C)O[C@@H](C)C5)cc4)n[nH]c3c1)C(=O)N2]
or a pharmaceutically acceptable salt thereof, and
(ii) an effective amount of an immune checkpoint inhibitor wherein the immune checkpoint inhibitor is a PD-1 inhibitor or a PD-L1 inhibitor.

2. The method according to claim 1, wherein the subject in need thereof is administered Compound (I) in an amount of 3 mg to 96 mg, or a pharmaceutically acceptable salt thereof in an amount equivalent to 3 mg to 96 mg of Compound (I).

3. The method according to claim 2, wherein the subject in need thereof is administered a 1:1 fumarate salt of Compound (I), wherein the molar ratio between compound (I) and fumaric acid is 1:1.

4. The method according to claim 3, wherein the immune checkpoint inhibitor is a PD-1 inhibitor.

5. The method according to claim 4, wherein the PD-1 inhibitor is pembrolizumab, nivolumab, cemiplimab (REGN2810), spartalizumab (PDR001), camrelizumab (SHR1210), Sintilimab (IBI308), Tislelizumab (BGB-A317), Toripalimab (JS001), Nivolumab (BMS-936558), AMP-224, or AMP-514.

6. The method according to claim 4, wherein the PD-1 inhibitor is pembrolizumab, nivolumab, or cemiplimab (REGN2810).

7. The method according to claim 3, wherein the immune checkpoint inhibitor is a PD-L1 inhibitor.

8. The method according to claim 7, wherein the PD-L1 inhibitor is atezolizumab, avelumab, durvalumab, JS003, KN035, CK-301, AUNP12, CA-170, BMS-936559, BMS-986189, or SHR-1316.

9. The method according to claim 7, wherein the PD-L1 inhibitor is atezolizumab, avelumab, or durvalumab.

10. The method according to claim 7, wherein the PD-L1 inhibitor is durvalumab.

11. The method according to claim 10, wherein the triple negative breast cancer is unresectable or metastatic.

12. The method according to claim 11, wherein the subject in need thereof is administered Compound (I) or a pharmaceutically acceptable salt thereof once daily for a week.

13. The method according to claim 11, wherein the subject in need thereof is administered Compound (I) or a pharmaceutically acceptable salt thereof in a cycle of once daily for a week and then a week off, wherein the cycle is repeated at least once.

14. The method according to claim 11, wherein the subject in need thereof is administered the checkpoint inhibitor once every four weeks.

15. The method according to claim 11, wherein the subject in need thereof is administered Compound (I) in an amount of 32 mg to 64 mg, or a pharmaceutically acceptable salt thereof, in an amount equivalent to 32 mg to 64 mg of Compound (I).

16. The method according to claim 11, wherein the subject in need thereof is administered Compound (I) in an amount of 32 mg, or a pharmaceutically acceptable salt thereof in an amount equivalent to 32 mg of Compound (I).

17. The method according to claim 11, wherein the subject in need thereof is administered Compound (I) in an amount of 40 mg, or a pharmaceutically acceptable salt thereof in an amount equivalent to 40 mg of Compound (I).

18. The method according to claim 11, wherein the subject in need thereof is administered Compound (I) in an amount of 48 mg, or a pharmaceutically acceptable salt thereof in an amount equivalent to 48 mg of Compound (I).

19. The method according to claim 11, wherein the subject in need thereof is administered Compound (I) in an amount of 64 mg, or a pharmaceutically acceptable salt thereof in an amount equivalent to 64 mg of Compound (I).

20. The method according to claim 11, wherein the subject in need thereof is administered durvalumab in an amount of 1500 mg when the subject is > 30 kg in weight or in an amount of 20 mg/kg when the subject is ≤ 30 kg.
